(19) United States
(12) Patent Application Publication
(10) Pub. No.: US 2009/0159841 A1
(43) Pub. Date: Jun. 25, 2009

(54) Recovery of Optically Active Epoxy Alcohols

(75) Inventors: Ann Marie Elderidge, King of Prussia, PA (US); Christopher Werner, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION, Philadelphia, PA (US)

(21) Appl. No.: 11/569,897

(22) PCT Filed: Jun. 9, 2005
(86) PCT No.: PCT/US2005/020395

§ 371 Date: Dec. 1, 2006

Related U.S. Application Data
(60) Provisional application No. 60/578,587, filed on Jun. 10, 2004.

(51) Int. Cl. C09K 3/00; C07D 301/32; C07D 405/02; C07D 301/19

(52) U.S. Cl. 252/182.12; 549/541; 548/467; 549/529

(57) ABSTRACT

A process to recover optically active epoxy alcohols from asymmetric epoxidation reaction mixtures such as those produced using the Sharpless method of epoxidation of allylic alcohols. The process includes adding a reducing agent to reduce an organic hydroperoxide in the asymmetric epoxidation reaction product to a corresponding alcohol to form a reduced epoxidation reaction mixture; adding the reduced reaction product to a film evaporation unit to form a residue fraction and an optically active epoxy alcohol distillate fraction; and distilling the optically active epoxy alcohol distillate fraction to purify the optically active epoxy alcohol.

### FIELD OF THE INVENTION

This invention is directed to a process to recover optically active epoxy alcohols from reaction mixtures containing an asymmetric catalyst system. More particularly, the invention is directed to methods of recovering (2S,3R)-1,2-epoxy-4-penten-3-ol from a reaction mixture comprising (2S,3R)-1,2-epoxy-4-penten-3-ol, an organic hydroperoxide, a transition metal, chiral ligand complex and a reaction solvent.

### BACKGROUND OF THE INVENTION

Optically active (non-racemic) epoxy alcohols are versatile starting materials and intermediates in the synthesis of chiral natural products and their derivatives. Many optically active compounds prepared from optically active epoxy alcohols have a high physiological activity. The synthetic utility of non-racemic epoxy alcohols has been extensively reviewed in Hanson, Chemical Reviews 91(4), 437-473 (1991).

Commercial scale preparation of optically active epoxy alcohols from inexpensive racemic starting materials may be carried out using an asymmetric epoxidation system developed by Dr. K. Barry Sharpless and co-workers. In the Sharpless process, an allylic alcohol reacts with an organic hydroperoxide in the presence of a titanium/chiral complex catalyst. Although the Sharpless process provides good yields of optically active epoxy alcohols with relatively high enantioselective excess, the recovery of the epoxy alcohol from the resulting epoxidation reaction mixture raises problems for large-scale commercial production.

In particular, 1,2-epoxy-4-penten-3-ol is desirable because it is a useful precursor in the synthesis of compounds of medicinal value. For example, Alex Romero and Chi-Huey Wong disclose synthesis of 1,2-epoxy-4-penten-3-ol as an intermediate in the preparation of australine and 7-epialexine, and suggest it may be used in the synthesis of other stereoisomers and analogues related to the hydroxylated pyrrolizidine class of alkaloids. A. Romero and C. Wong, J. Org. Chem. (2000), 65:8264-8268. The synthesis of 1,2-epoxy-4-penten-3-ol is also desirable as an intermediate in the preparation of the inhibitors of the cathepsin family of cysteine proteases that are reported in WO 01/70232, which inhibitors are useful for treating osteoporosis, peridontitis and arthritis.

Simple distillation results in considerable loss of the desired epoxy alcohols, which are known to be unstable and reactive. For example, attempts to vacuum distill optically active epoxy alcohols from the Sharpless reaction product mixture can result in significant amounts of polymerized product. Romero and Wong describe an isolation process that includes treatment of the reaction mixture with an aqueous Na₂SO₄ solution, followed by filtration and silica gel chromatography. However, the reaction mixture may filter very slowly and the chromatography can be difficult to scale up to commercial amounts. U.S. Pat. No. 5,288,882 discloses contacting the reaction mixture with a reducing agent, polyalcohol, or both to inhibit the epoxidation catalyst or reduce the hydroperoxide before distillation. Yet even after this treatment, the distillation step can still result in significant loss of yield due to polymerization or non-stereoselective epoxidation reactions occurring during recovery. As a result, improved methods to recover optically active epoxy alcohols from asymmetric epoxidation reaction mixtures with minimal loss would be of considerable value.

### SUMMARY OF THE INVENTION

The invention is directed to a process to recover an optically active epoxy alcohol from an asymmetric epoxidation reaction mixture containing the optically active epoxy alcohol, an organic hydroperoxide and a transition metal-chiral ligand complex epoxidation catalyst. The process of the invention includes: (a) contacting the asymmetric epoxidation reaction mixture with a reducing agent such that substantially all of the organic hydroperoxide is reduced to produce a reduced epoxidation reaction mixture; (b) adding the reduced epoxidation reaction mixture to a film evaporation unit in which the reduced epoxidation reaction mixture is separated to form a residue fraction and an optically active epoxy alcohol distillate fraction; and (c) distilling the optically active epoxy alcohol distillate fraction to produce a purified optically active epoxy alcohol.

The invention is also directed to a method of making an optically active epoxy alcohol. The method of the invention includes contacting an allylic alcohol with an organic hydroperoxide and a transition metal-chiral ligand complex epoxidation catalyst in a reaction solvent under conditions to produce the optically active epoxy alcohol; adding a reducing agent such that substantially all of the organic hydroperoxide is reduced to produce a reduced epoxidation reaction mixture; concentrating the reduced epoxidation reaction mixture by removal of at least some of the reaction solvent to produce a concentrated epoxidation reaction product; separating the concentrated epoxidation reaction product in a film evaporation unit to form a residue fraction and an optically active epoxy alcohol distillate fraction; and distilling the optically active epoxy alcohol distillate fraction to produce a purified optically active epoxy alcohol.

The method of the invention can further include adding an azeotropic solvent prior to or after the adding of the reducing agent. The azeotropic solvent forms an azeotrope with the optically active epoxy alcohol. Alternatively, the azeotropic solvent can be added following concentration of the reduced epoxidation reaction mixture.

The invention is also directed to a composition comprising at least 70% by weight of an optically active epoxy alcohol, less than 15% by weight of an azeotropic solvent, and 0.05% to 2% by weight of cumene alcohol.

### DETAILED DESCRIPTION OF THE INVENTION

Applicants discovered a process to purify optically active epoxy alcohols produced using the Sharpless asymmetric epoxidation system. The term “optically active” as used herein means that the quantity of one enantiomer, e.g., (R), is greater than that of the other, e.g., (S), or visa versa, in a product mixture. The optically active epoxy alcohols used in the present invention are prepared using a Sharpless asymmetric catalyst or an analogue or derivative thereof. Optically active epoxy alcohols prepared in this manner typically have enantiomeric excess greater than 90%, and the reaction often provides product yields of greater than 85%. However, as stated earlier, a major disadvantage of using the Sharpless process is the difficulty in recovering the desired, optically active epoxy alcohol from the asymmetric epoxidation reaction mixture.

The loss of epoxy alcohol due to polymerization, thermal decomposition, and hydrolysis during recovery can be significant. For example, in the preparation of (2S,3R)-1,2-epoxy-4-penten-3-ol, product losses exceeding 60% were observed during recovery using conventional distillation. The asymmetric epoxidation reaction provided product yields greater than 85%, as measured by gas chromatography; however, the recovered yield was only about 35% after recovery using conventional vacuum distillation. The present invention is directed to minimizing loss of the epoxy alcohol during subsequent purification.

The process of the invention can recover yields of (2S,3R)-1,2-epoxy-4-penten-3-ol of about 50% or greater, preferably of about 60% or greater, as measured by gas chromatography. In contrast, the use of vacuum distillation at a pot temperature of about 40° C. to about 100° C. and a reduced pressure of about 40 to 60 mm Hg(a) provided yields of about 32% as measured by gas chromatography.

The Sharpless process for the preparation of optically active alcohols is described in U.S. Pat. Nos. 4,471,130, 4,764,628, and 4,594,439, the entire disclosures of which are incorporated herein by reference. The review by Finn et al. in Asymmetric Synthesis, Morrison, ed., Academic Press, New York (1985), Vol. 5, Chapter 8, p. 247 also provides insight into the Sharpless process. In brief, the Sharpless process involves the use of an organic hydroperoxide as an oxygen source, an allylic alcohol as a substrate, and a transition metal catalyst complexed to a chiral ligand. The organic hydroperoxide is typically a secondary or tertiary aliphatic or aromatic hydroperoxide such as t-butyl hydroperoxide, t-amyl hydroperoxide, cumene hydroperoxide, ethyl benzene hydroperoxide, cyclohexyl hydroperoxide, and triphenylmethyl hydroperoxide. The use of cumene hydroperoxide is preferred. The transition metal in the catalyst is preferably selected from titanium, molybdenum, zirconium, vanadium, tantulum, and tungsten, with titanium being preferred. Suitable chiral ligands are disclosed in the art. Particularly preferred chiral ligands are chiral alcohols, such as chiral glycols (dihydric alcohols). More particularly preferred are ester and amide derivatives of tartaric acid.

Preferably, the Sharpless process occurs in the presence of an inert organic solvent. The organic solvent used in the Sharpless process is selected so as to provide rapid and enantioselective conversion of the allylic alcohol to the optically active epoxy alcohol. Preferred solvents for use include halogenated hydrocarbons such as methylene chloride, dichloroethane, carbon tetrachloride. Aliphatic hydrocarbons such as hexane, isooctane, cyclohexane, as well as aromatic hydrocarbons such as toluene, ethyl benzene, and cumene can also be used.

The asymmetric epoxidation is typically carried out with a stoichiometric excess of the organic hydroperoxide relative to the allylic alcohol. Following the asymmetric epoxidation reaction, the asymmetric epoxidation reaction mixture is contacted with a reducing agent to reduce the organic hydroperoxide remaining in the reaction product. The neutralizing of the excess organic hydroperoxide in the reaction product occurs for two reasons. First, the distillation of mixtures containing peroxides raises a significant safety issue. Second, the presence of hydroperoxide in the reaction product can lead to undesirable by-products, making recovery of the epoxy alcohol more difficult. For example, in U.S. Pat. No. 5,288,882, it has been noted that the presence of hydroperoxide in the reaction product leads to additional epoxidation of the unreacted allylic alcohol during distillation. Because this epoxidation occurs at higher temperatures, however, the additional epoxidation is much less stereoselective than the initial epoxidation.

Preferably, the excess organic hydroperoxide is neutralized by contacting the asymmetric reaction product with a reducing agent selected from sulfur(II) compounds, sulfur(III) compounds, or phosphorous(III) compounds to completely reduce any excess hydroperoxide present to the corresponding alcohol. These reducing agents do not adversely interact with optically active epoxy alcohol, which are known to be highly susceptible to degradation. Typically, a slight excess of the reducing agent is used relative to an estimated amount of hydroperoxide present to assure complete hydroperoxide reduction. Some sulfur(II) and sulfur(III) compounds that can be used include both organic and inorganic compounds. These include, for example, alkali metal salts of hydrogen sulfites (HSO₃M), sulfites (SO₃M₂), and disulfites (HS₂O₃M and M₂S₂O₅). Organic sulfides and organic sulfoxides including, for example, dibenzyl sulfoxide, dibutyl sulfoxide, dimethyl sulfoxide, 4,4′-ditolylsulfoxide, can also be used.

The preferred reducing agents used in the process of the invention are phosphorous(III) compounds. These include organic phosphines having the general structure R₁R₂R₃P wherein R₁, R₂, and R₃ are the same or different and are hydrocarbon groups such as alkyl, aryl, and aryl alkyl (e.g., triphenylphosphine, triethylphosphine, diphenylethylphosphine). The use of organic phosphites is particularly preferred. These compounds have the general structure R₁OP(OR₂)OR₃ wherein R₁, R₂ and R₃ are as described above. Example compounds include trimethyl phosphite, triethyl phosphite, tri-isopropylphosphite, triphenylphosphite, and tri(4-tolyl)phosphite).

The most preferred reducing agent used in the process of the invention is trimethyl phosphite.

Following the Sharpless reaction process, and preferably after reduction of excess peroxide, preferably the bulk of the reaction solvent, typically dichloromethane, is removed, preferably under reduced pressure. This step can be carried out using methods well known in the art such as rotary evaporation or simple distillation. However, because a film evaporation unit is required for subsequent steps in the process of the invention, a film evaporation unit can also be used to remove the bulk of the solvent. The film evaporation units used in the process of the invention include wiped-film evaporation (WFE) units and falling-film evaporation units.

A WFE unit includes a heated body into which a liquid material is charged. The feed system is typically a gravity feed addition flask or a positive displacement pump. A wiper blade or roller assembly rotating at a predetermined speed spreads the liquid into a thin film along the sides of the heated body. The film progresses down through the inside body wall aided by gravity and slots in the wiper blades or rollers. The non-evaporated material or WFE residue flows out of the system continuously. The material that distills is condensed with an interior condenser (within the heated body), an exterior condenser, or both depending on the type of WFE design used.

The WFE unit is ideal for the distillation of heat sensitive materials. Rather than continually heating the bulk sample in a pot distillation over a relatively long period, only the sample along the heated body is heated. This can reduce the contact time of the epoxy alcohol at elevated temperatures from hours to minutes. This design significantly reduces the amount of time the desired product is in contact with elevated temperatures. The feed rate and the wiper speed are used to control the time the material is in the still body.

For large quantities of material, a series of metering pumps can be used to pump in feed material at a reproducible, controlled rate. Two receiver pumps can be used per stage to remove both the bottom product and the distillate. With instrumentation and controls installed on the units, there is the ability to adjust the distillation parameters of vacuum level, temperature and wiper speed.

The WFE unit can be configured as a two-stage unit. As a two-stage process, typically the first stage is used for the removal of low-boiling components followed by molecular distillation of the desired product in the second stage. The flashing off of low boilers in the first stage can usually be accomplished more rapidly than the actual distillation of the product, which is achieved in the second stage. The efficiency of the first stage is improved by larger feed and receiver flasks or pumps, extra condensing power as given by an external condenser and higher heating capacity.

The present invention may be performed for example in WFE units manufactured by Lybold-Heraeus and Pope Scientific, Inc., or modified units from these manufacturers. For example, incorporation of an additional condenser within the heated body will maximize condenser surface area, which can lead to more efficient recovery of distillate. The incorporation of an internal condenser within a WFE unit is defined as a molecular still. The term WFE unit also includes a molecular still unit. A general discussion of WFE units can be found in the publication: “Agitated Thin-Film Evaporators: A Three Part Report”, Parts 1 to 3; A. B. Mutzenburg, N. Parker and R. Fischer; Chemical Engineering, Sep. 13, 1965.

Following removal of the bulk of the reaction solvent from the reduced epoxidation reaction mixture, it is preferred that an azeotropic solvent be added to the concentrated product and the diluted product solution then be passed through a film evaporation unit. Of course, the azeotropic solvent can be added at any point in the initial stages of the process including to the asymmetric epoxidation reaction mixture, the reduced epoxidation reaction mixture, or the concentrated asymmetric reaction product. The azeotropic solvent forms a minimum boiling heterogeneous azeotrope with the epoxy alcohol.

For purification of epoxy alcohols that are relatively low boiling, the azeotropic solvent will generally have a boiling point at 760 mm Hg from about 150° C. to 220° C. The term “boiling point” also includes a set boiling range of a mixture of high boiling compounds. Relatively inexpensive high boiling solvents or solvent mixtures can be used depending upon the contaminant constraints on subsequent steps in the synthetic process. Preferred azeotropic solvents are selected from aromatic hydrocarbons, halogenated aliphatic hydrocarbons and aliphatic hydrocarbons. Preferred are decahydronapthalene, trimethylbenzene, ethyltoluene, t-butyl cyclohexane, cyclooctane, and C₁₀-C₁₂ straight or branched saturated hydrocarbons. For the purification of 1,2-epoxy-4-penten-e-ol, the preferred azeotropic solvent is a mixture of (cis) and (trans)decahydronaphthalenes sold under the trade name Decalin®. The azeotropic solvent used in the process depends upon the boiling points of the other components in the system, i.e., the epoxy alcohol, reducing agent, and oxidizing agent.

The azeotropic solvent should be non-reactive towards the epoxy alcohol under the distillation conditions used and preferably has a miscibility with the epoxy alcohol such that that one or more collected distillation fractions containing azeotrope solvent and epoxy alcohol separate into two distinct liquid phases after condensing from the vapor state. One phase is relatively rich in the epoxy alcohol (typically, at least 80% by weight) whereas the other is relatively lean in the epoxy alcohol, i.e., is comprised predominantly of the azeotropic solvent.

The amount of azeotropic solvent used in the process of the invention is dependent upon the amount of epoxy alcohol to be removed by azeotropic distillation and the related proportion of each component in the azeotrope. As these factors are known or easily measured by standard methods, the minimum amount of azeotropic solvent required for optimal recovery can be readily calculated.

After addition of the azeotropic solvent the now diluted product solution is passed through a film evaporation unit. Alternatively, after reduction of the organic hydroperoxide the product mixture can be added to a film evaporation unit without the addition of an azeotropic solvent. Typically, for liquid, low boiling epoxy alcohols, the film evaporation unit is a WFE unit operated at reduced pressures and jacket temperatures below 120° C. to minimize decomposition of product. The low and medium boiling components are removed as distillate leaving the epoxidation catalyst, polymeric materials, and other higher boiling components in the WFE residues.

The epoxy alcohol azeotropes with the azeotropic solvent and is collected as distillate fractions. These distillate fractions preferably are subjected to batch distillation for further purification. Following batch distillation, the distillate fractions containing the epoxy alcohol and the azeotropic solvent form two layers that preferably can be conveniently separated. The azeotropic solvent phase can be returned to the distillation system for efficient use of the azeotropic solvent and to minimize product loss to this phase.

Because azeotropes containing epoxy alcohol are relatively high boiling, and epoxy alcohols are relatively unstable to elevated temperatures, a batch distillation of the collected distillate fractions is typically carried out at reduced pressures e.g., from 0.1 to 100 mm Hg(a), preferably from 0.1 to 45 mm Hg(a). The pressure should be adjusted so as to provide an azeotrope boiling point, i.e., the temperature of the vapor taken overhead, between about 25° C. to 125° C. Preferably, the pot (bottoms) temperature does not at any point exceed 120° C. to minimize decomposition and polymerization of the epoxy alcohol.

The term “batch” distillation is defined as the distillation of distillate fractions. The batch distillation can be carried out with a WFE unit. However, to obtain higher purity epoxy alcohol product mixtures, batch distillation is carried out using conventional vacuum distillation techniques known in the art. Conventional distillation columns of any configuration can be utilized, preferably columns having from 5 to 60 theoretical contacting stages. The distillation columns should also operate with reflux ratios of from 0.5 to 15, preferably from 1 to 6.

The invention is also directed to a composition produced using the inventive methods. In one embodiment, the composition is produced from the Sharpless epoxidation of allylic alcohols using cumene hydroperoxide as the organic hydroperoxide. The composition contains an optically active epoxy alcohol and cumene alcohol. The composition of the invention comprises at least 70% by weight of optically active epoxy alcohol, less than 15% by weight of an azeotropic solvent, more preferably 0.1% to 15% by weight of an azeotropic solvent, and 0.05% to 2% by weight of cumene alcohol. Preferably, the enantioselective excess of the optically active, epoxy alcohol is greater than 80%, more preferably greater than 90%. The composition of the invention can also contain small amounts of the oxidation product of the reducing agent, such as trimethyl phosphate.

The preferred composition of the invention will comprise at least 80% by weight of optically active epoxy alcohol, 0.1% to 10% by weight of an azeotropic solvent, and 0.05% to 2%, 0.1% to 1%, or 0.1% to 0.6% by weight of cumene alcohol.

The optically active epoxy alcohol in the methods of the invention is preferably

(2S,3R)-1,2-epoxy-4-penten-3-ol, which is present in enantioselective excess. Preferably, the enantioselectivity excess is greater than 80%, more preferably greater than 90%.

The method of recovery of the optically active, (2S,3R)-1,2-epoxy-4-penten-3-ol product includes adding to the asymmetric epoxidation reaction mixture a reducing agent to convert organic hydroperoxide to the corresponding alcohol; optionally concentrating the resulting reduced epoxidation reaction mixture by removal of at least some of the reaction solvent; optionally adding an azeotropic solvent that forms an azeotrope with 1,2-epoxy-4-penten-3-ol; directing the resulting mixture to a film evaporation unit to form a residue fraction and an optically active, (2S,3R)-1,2-epoxy-4-penten-3-ol distillate fraction; and distilling the optically active, (2S,3R)-1,2-epoxy-4-penten-3-ol distillate fraction to form purified optically active, (2S,3R)-1,2-epoxy-4-penten-3-ol. Preferably, the reducing agent is trimethyl phosphite, the organic hydroperoxide is cumene hydroperoxide, and the azeotropic solvent is Decalin®. The oxidized by-product, i.e., trimethyl phosphate, does not adversely impact the recovery of the optically active epoxy alcohol. Furthermore, the trimethyl phosphate by-product is believed to provide an intermediate boiling point component between epoxy alcohol and cumene alcohol in a Decalin® azeotropic solvent system, enabling cumene alcohol levels to be kept below 2% in the epoxy alcohol product mixture. The use of an alternative reducing agent such as triethyl phosphite does not provide this intermediate boiling component, thus resulting in cumene alcohol concentrations in excess of 2.0%.

The invention is also directed to a method of making 2-[(1S)-1-(2R)-oxiranyl-2-propenyl]-1H-isoindole-1,3(2H)-dione comprising contacting an optically active, (2S,3R)-1,2-epoxy-4-penten-3-ol product mixture with phthalimide wherein the optically active, (2S,3R)-1,2-epoxy-4-penten-3-ol product mixture comprises at least 70% by weight optically active (2S,3R)-1,2-epoxy-4-penten-3-ol, less than 15% by weight of an azeotropic solvent, and 0.05% to 2% by weight of cumene alcohol.

The invention is also directed to a method of making benzofuran-2-carboxylic acid {(S)-3-methyl-1-[(4S,7R)-7-methyl-3-oxo-1-(pyridine-2-sulfonyl)-azepin-4-ylcarbamoyl]-butyl}-amide, the method comprising recovering (2S,3R)-1,2-epoxy-4-penten-3-ol from an asymmetric epoxidation reaction mixture comprised of (2S,3R)-1,2-epoxy-4-penten-3-ol, an organic hydroperoxide and a transition metal-chiral ligand complex epoxidation catalyst by separating an (2S,3R)-1,2-epoxy-4-penten-3-ol distillate fraction from a residue fraction using a film evaporation unit; distilling the (2S,3R)-1,2-epoxy-4-penten-3-ol distillate fraction to produce a purified (2S,3R)-1,2-epoxy-4-penten-3-ol, then proceeding as described in scheme 1 in WO 01/70232, or as described in U.S. Patent App. Ser. No. 60/31,949, filed Nov. 21, 2001, both of which applications are hereby incorporated herein in their entirety, to produce benzofuran-2-carboxylic acid ((S)-3-methyl-1-[(4S,7R)-7-methyl-3-oxo-1-(pyridine-2-sulfonyl)-azepin-4-ylcarbamoyl]-butyl}-amide. The optically active (2S,3R)-1,2-epoxy-4-penten-3-ol product mixture comprises at least 70% by weight optically active, (2S,3R)-1,2-epoxy-4-penten-3-ol, 0.1% to 15% by weight of an azeotropic solvent, and less than 2% by weight of cumene alcohol, preferably less than 1% by weight, more preferably less than 0.6% by weight, cumene alcohol.

The invention and its benefits will be better understood with reference to the following examples. These examples are intended to illustrate specific embodiments within the overall scope of the invention as claimed, and are not to be understood as limiting the invention in any way.

The following experiments concern parameters and optimal recovery options for the recovery of (2S,3R)-1,2-epoxy-4-penten-3-ol. The parameters investigated were the following: 1) the presence of Decalin® as an azeotrope agent; 2) the use of a WFE unit to provide WFE distillate fractions, prior to batch distillation; and 3) the type of reducing agent used to neutralize the hydroperoxide.

The following examples were all performed on an asymmetric epoxidation reaction mixture produced according to the following reaction.

### Preparation of Asymmetric Reaction Product.

The epoxidation of 3-hydroxy-1,4-pentadiene to (2S,3R)-1,2-epoxy-4-penten-3-ol is conducted in dichloromethane/4A sieves at −15° C. in the presence of a Sharpless asymmetric catalyst system comprising (Ti(OiPr)₄, D(−)diisopropyl tartrate (“DIPT”), and cumene hydroperoxide, as shown in Scheme 1.

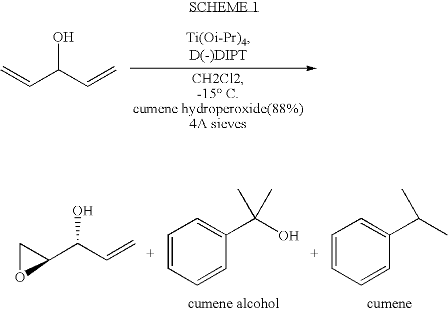

The Sharpless asymmetric reaction process provides a product yield of the epoxy alcohol, as measured by gas chromatography (“gc”), of about 82% to 90% with an enantiomeric excess greater than 80%.

### Comparative Example 1

### Azeotropic Batch Distillation without WFE Treatment

A slight molar excess of trimethyl phosphite is added to the asymmetric reaction solution to convert an estimated amount of the remaining cumene hydroperoxide to cumene alcohol. The reaction mixture is filtered to remove molecular sieves and is concentrated under reduced pressure to remove the bulk of the dichloromethane solvent. Three volumes of Decalin® are added to the asymmetric reaction product. The resulting solution is charged to a distillation flask and an azeotropic distillation is carried out with a 10 tray Oldershaw column at a reflux ratio of 3. A WFE unit is not used in this recovery process (Table 4, Ex. 1). During the distillation, the pot temperature ranged from 40° C. to 100° C. and attempts were made to maintain a process pressure between about 40 to 60 mm Hg(a).

Distillation operations were successful in purifying the product to 69% GC Peak Area Ratio (GC PAR) and reducing cumene alcohol to less than 3%, but generated only a recovered 32% yield (GC PAR). The low yield has been attributed to polymerization of the epoxy alcohol in the pot during the distillation as the epoxy alcohol is exposed to elevated temperatures, high boiling reagents, and catalyst during the distillation.

### Example 2

### Azeotropic WFE and Batch Distillation Process with Decalin®

A slight molar excess of trimethyl phosphite is added to the asymmetric reaction solution to convert an estimated amount of the remaining cumene hydroperoxide to cumene alcohol. The reaction solution is filtered to remove the molecular sieves and is concentrated by removal of the bulk of the dichloromethane solvent at 80 to 90 mm Hg(a) and room temperature jacket conditions. Three volumes of decahydronapthalene, sold under the trade name Decalin®, is added to the concentrated reaction solution. The solution is charged to a WFE unit operating at a reduced pressure of 1 mm Hg(a), 50° C. evaporator chamber jacket temperature and −15° C. condenser temperatures, and equipped with an acetone/dry ice vacuum trap.

The collected WFE distillate fractions include the desired epoxy alcohol, Decalin®, and relatively small amounts of cumene alcohol. The collected WFE residue still contains 23% by gas chromatography of the theoretical yield of epoxy alcohol. Three volumes of Decalin® is added to this residue, which is then charged to the WFE unit. The second pass reduces loss of the epoxy alcohol to less than 2% by gas chromatography in the WFE residue. GC analytical results for the two WFE passes (presented in Tables 1 and 2 below) indicate that the use of a WFE unit is successful in isolating the optically active epoxy alcohol from the high boiling point DIPT, and reducing the cumene alcohol content of the epoxy alcohol product mixture.

The epoxy alcohol rich WFE distillate fractions, i.e., fractions from the coldfinger, external condenser and vacuum trap, are collected and charged to a distillation flask to remove additional low boiling solvents, trace amounts of catalyst, cumene, trimethyl phosphate, and cumene alcohol, and to partially separate the azeotropic solvent from the optically active epoxy alcohol. The batch distillation is conducted in a standard round bottom reaction vessel equipped with a 10 tray Oldershaw column operating at a pressure of 25 to 45 mm Hg(a) and a reflux ratio of 3.0. Condensate fractions are collected over the course of distillation operations. The condensate fractions exhibit a two phase distillate from which epoxy alcohol is isolated as a heavy phase while the Decalin® phase is returned to the distillation flask.

TABLE 1
Wiped Film Evaporator (WFE) - Pass I

| | | Sample | Epoxy | Decalin | Cumene | | | | |
|---|---|---|---|---|---|---|---|---|---|
| | | Weight | Alcohol | (trans & cis) | Alcohol | Cumene | (MeO)₃PO | IPA | Other |
| Fraction | Phase | (g) | % PAR | % PAR | % PAR | % PAR | % PAR | % PAR | % PAR |
| Coldfinger | Bottom | 70.6 | 26.7% | 37.1% | 32.2% | 1.3% | 2.6% | — | 0.1% |
| | Top | 125.4 | 4.4% | 84.8% | 8.9% | 1.5% | 0.4% | — | — |
| Ext. Condenser | Bottom | 12.8 | 41.5% | 25.8% | 26.6% | 1.8% | 3.3% | 0.5% | 0.5% |
| | Top | 74.0 | 2.7% | 91.1% | 3.8% | 2.2% | — | — | 0.2% |
| Vac. Trap | Single Phase | 153.0 | 7.6% | 58.7% | 1.5% | 8.9% | — | 20.1% | 3.2% |
| Residues | Single Phase | 252.4 | 8.1% | 10.6% | 74.3% | — | 3.8% | — | 3.2% |

TABLE 2
Wiped Film Evaporator (WFE) Pass II

| | | Sample | Epoxy |
|---|---|---|---|
| | | Weight | Alcohol |
| Fraction | Phase | (g) | % PAR |
| Coldfinger | Bottom | 306.5 | 3.7% |
| Ext. Condenser | Bottom | 40.3 | 29.0% |
| Vac. Trap | Negligible | — | — |
| Residues | Single Phase | 103.8 | 1.5% |

Table 3 below provides the gas chromatograph analytical data for each of the distillate fractions collected. A weight based assay conducted on a composite of product fractions 4, 5 and 6 indicate a 72% isolated yield. This is a substantial improvement over the 35% yields obtained without the WFE unit in Comparative Example 1. The purity of the (2S,3R)-1,2-epoxy-4-penten-3-ol, i.e., w/wt of (2S,3R)-1,2-epoxy-4-penten-3-ol in the collected fraction, is also increased from less than 65% to over 81% purity with cumene, trimethyl phosphate, and Decalin® constituting the principal contaminants. The results of the chiral assay of (2S,3R)-1,2-epoxy-4-penten-3-ol obtained from the wiped film evaporation procedure show less than 0.5% undesired isomer (limit of detection).

TABLE 3

| | | Sample | Epoxy | Decalin | Cumene | | | | Starting | |
|---|---|---|---|---|---|---|---|---|---|---|
| | | Weight | Alcohol | (trans & cis) | Alcohol | Cumene | (MeO)₃PO | IPA | Material | Other |
| Fraction | Phase | (g) | % PAR | % PAR | % PAR | % PAR | % PAR | % PAR | % PAR | % PAR |
| Fraction-1: | Single Phase | 71.5 | 5.3% | 37.0% | 0.6% | 3.2% | — | 51.4% | 1.4% | 1.1% |
| Fraction-2: | Single Phase | 34.3 | 1.4% | 1.6% | 0.4% | 0.9% | — | 75.0% | 10.7% | 10.0% |
| Fraction-3: | Single Phase | 4.8 | 1.2% | 7.9% | — | 12.7% | — | 67.4% | 6.8% | 4.0% |
| Fraction-4: | Bottom | 40.9 | 76.4% | 14.8% | 6.0% | 4.7% | — | 2.4% | 1.5% | — |
| Fraction-5: | Bottom | 25.9 | 89.6% | 5.6% | 0.4% | 3.9% | 0.5% | — | — | 0.0% |
| Fraction-6: | Bottom | 13.9 | 55.1% | 1.8% | 0.8% | — | 37.2% | — | — | 5.1% |
| Pot Residues | Single Phase | 613.2 | 0.4% | 85.6% | 13.7% | — | — | — | — | 0.3% |

Isolated Yield = 72.6% based on weight based assay for composite of fractions 4, 5 and 6.
Product Assay = 81% for composite of fractions 4, 5 and 6.

The results of Example 2 demonstrate that the use of a WFE unit to separate the epoxy alcohol from the reduced epoxidation reaction mixture, and in particular to separate from the DIPT catalyst and high boiling point components that contribute to polymer formation during subsequent batch distillation operations, provides a significant improvement in recovery yields.

### Examples 3 to 5

Similar procedures are used according to Example 2 with the exception that the batch distillation is refined by further limiting the product cut temperature range to reduce carry over of cumene and trimethyl phosphate in the final optically active epoxy alcohol product. These experiments (Table 4, Exs. 3 to 5) demonstrate that weight based product assays of the epoxy alcohol can be raised to 90% but at a substantial impact to overall product yield.

### Examples 6 and 7

### Azeotropic WFE with Triethyl Phosphite

Similar procedures are used according to Example 2 with the exception that triethyl phosphite is used instead of trimethyl phosphite (Table 4, Exs. 6 and 7). The product fractions obtained from these operations generate product yields of 52% and 56% and product assays as high as 94%, but both fractions contain 1.9% cumene alcohol. This level of cumene alcohol is substantially higher then the distillate cuts of Examples 2-5. It is believed that the observed differences in contaminate levels is due to the absence of an intermediate boiling point component, i.e., trimethyl phosphate.

### Example 8

### WFE and Batch Distillation without Decalin®

Similar procedures are used according to Example 2 with the exception that Decalin® is not added to the reaction solution. In other words, the distillations are carried out without an azeotropic solvent present to evaluate if an azeotropic solvent is necessary to facilitate epoxy alcohol removal from the asymmetric reaction product (Table 4, Ex. 8). Three passes through the WFE unit are required to achieve comparable product fractions with comparable purity levels. The third pass requires lower pressure conditions (0.2 mm Hg (a)) than the pressure conditions in the other Examples (0.5 to 0.6 mm Hg (a)). The WFE operations achieved a typical 50% reduction in cumene alcohol weight compared to Comparative Example 1. However, the WFE distillate produced in the absence of Decalin® is tinged yellow (the WFE distillate is typically transparent). Also, the WFE distillate from the third pass contained over 1% DIPT by GC PAR preventing its use in subsequent distillation operations, which accounts for an additional 2.7% loss in yield for the process.

Batch distillation operations conducted in the absence of Decalin® results in product fractions with a 94% GC PAR. This is higher then the 88% PAR assay achieved with Decalin® due to elimination of the 5 to 7% PAR Decalin® contamination in the product fraction of Example 2. However Decalin®, a relatively inert solvent, does not generally interfere with subsequent synthetic reaction steps. In the absence of Decalin®, the cumene alcohol content is in excess of 2.0% PAR. The presence of too high a concentration of cumene alcohol in the optically active epoxy alcohol product can interfere with a subsequent step in the synthesis of a target compound. The product fractions also contain upwards of 0.2% DIPT by GC PAR, which is substantially higher then the non-detection levels typically observed in Example 2. The presence of too high a concentration of DIPT in the optically active epoxy alcohol product can interfere with a subsequent step in the synthesis. There was also evidence of polymer-like material at the pot residue liquid line, similar to what was observed with Comparative Example 1.

### Example 9

### WFE and Decalin® Charged Following WFE Operations

The collected batch distillate fractions and residue material from Example 8 are combined, charged with three volumes of Decalin® and charged to a batch distillation operation (Table 4, Ex. 9). The epoxy alcohol product fractions collected contain DIPT levels an order of magnitude lower then those conducted in the absence of Decalin® (Example 8), but higher then those observed if Decalin® is charged prior to WFE operations as in Example 2. Again, polymer-like material is observed over the course of the batch distillation operation.

Table 4 summarizes the results of Comparative Example 1 and Examples 2 to 9.

TABLE 4
Summary of WFE/Batch Distillation Results
Table 4: Summary of WFE/Batch Distillation Results

| | | Epoxy | Cumene | |
|---|---|---|---|---|
| | Isolated | Alcohol | Alcohol | |
| Experiment | Yield | w/w | % PAR | Description |
| 1 | 32.1% | 69.5% | 2.80% | Distillation without WFE |
| | | | | treatment |
| 2 | 72.6% | 82.0% | 0.17% | Initial WFE & batch |
| | | | | distillation with Decalin ® |
| 3 | 64.3% | 87.1% | 0.17% | Narrower collection |
| | | | | range to improve purity |
| 4 | 53.7% | 90.8% | 1.1% | Narrow collection range; |
| | | | | leak in reactor |
| 5 | 57.4% | 88.0% | 0.4% | Narrow collection range |
| 6 | 52.0% | 81.0% | 1.9% | Triethyl phosphite reduced |
| | | | | material |
| 7 | 56.0% | 94.1% | 1.9% | Triethyl phosphite reduced |
| | | | | material |
| 8 | 56.0% | 94.4% | 2.4% | WFE & distillation without |
| | | | | Decalin ® |
| 9 | 47.2% | 83.9% | 0.2% | Above with Decalin ® for |
| | | | | distillation ops. |

*Epoxy alcohol value for Example 9 is based on GC PAR, not w/w.

### Example 10

### Use of WFE for Removal of Solvent

A slight molar excess of trimethyl phosphite is added to the asymmetric reaction solution to convert an estimated amount of the remaining cumene hydroperoxide to cumene alcohol. The reaction mixture is filtered to remove molecular sieves. The filtered reaction solution is charged to a wiped-film evaporation (WFE) unit to concentrate the reaction solution by removal of the bulk of the dichloromethane solvent at around 85 mm Hg(a) and room temperature jacket conditions. Concentration of the reaction solution with the WFE unit provides solution yield losses of 10% based on GC PAR. Concentration operations conducted via rotavap or batch distillation typically generated a similar yield loss.

### Example 11

### Synthesis of 2-[(1S)-1-(2R)-oxiranyl-2-propenyl]-1H-isoindole-1,3(2H)-dione

To a toluene solution of triphenylphosphine (5.85 g, 1.05 equiv) is added phthalimide (1.03 eq). A toluene solution of diethyldiazocarboxylate (DIAD, 4.34 mL, 1.05 equiv) and (2S,3R)-1,2-epoxy-4-penten-3-ol product mixture (2.2 g of the epoxy alcohol, 1.0 eq) is added by syringe pump over 2.5 h at ambient temperature. The reaction is stirred for 1 h, chilled to 0° C. and t-butyl methyl ether added. After 1 h, the mixture is filtered and the filtrate concentrated to an oil. The resulting oil is dissolved in toluene and cooled to −25° C. overnight. The mixture is filtered and the filtrate concentrated. The product is then subjected to chromatography to obtain relatively clean fractions of the product.

What is claimed is:

1. A process to recover an optically active epoxy alcohol from an asymmetric epoxidation reaction mixture comprised of the optically active epoxy alcohol, an organic hydroperoxide and a transition metal-chiral ligand complex epoxidation catalyst comprising:
(a) contacting the asymmetric epoxidation reaction mixture with a reducing agent such that substantially all of the organic hydroperoxide is reduced to produce a reduced epoxidation reaction mixture;
(b) adding the reduced epoxidation reaction mixture to a film evaporation unit in which the reduced epoxidation reaction mixture is separated to form a residue fraction and an optically active epoxy alcohol distillate fraction; and
(c) distilling the optically active epoxy alcohol distillate fraction to produce a purified optically active epoxy alcohol.

2. The process of claim 1 wherein the reducing agent is trimethyl phosphite.

3. The process of claim 1 wherein the organic hydroperoxide is cumene hydroperoxide.

4. The process of claim 1 wherein the film evaporation unit is selected from the group consisting of a falling-film evaporation unit and a wiped-film evaporation unit.

5. The process of claim 1 further comprising adding an azeotropic solvent to the asymmetric epoxidation reaction mixture prior to contacting with a reducing agent, wherein the azeotropic solvent forms an azeotrope with the epoxy alcohol.

6. The process of claim 1 further comprising adding an azeotropic solvent to the film evaporation unit prior to or during the addition of the reduced epoxidation reaction mixture to the film evaporation unit, wherein the azeotropic solvent forms an azeotrope with the epoxy alcohol.

7. The process of claim 1 wherein the asymmetric epoxidation reaction mixture further comprises a reaction solvent and wherein, after contacting the mixture with a reducing agent and before adding the reduced epoxidation reaction mixture to the film evaporation unit, the reduced epoxidation reaction mixture is concentrated by removal of at least some of the reaction solvent.

8. The process of claim 1 wherein the optically active epoxy alcohol is (2S,3R)-1,2-epoxy-4-penten-3-ol.

9. The process of claim 8 wherein the reducing agent is trimethyl phosphite and the organic hydroperoxide is cumene hydroperoxide.

10. The process of claim 9 further comprising adding an azeotropic solvent to the film evaporation unit prior to or during step (b), wherein the azeotropic solvent forms an azeotrope with the epoxy alcohol.

11. The process of claim 10 wherein the azeotropic solvent is selected from the group consisting of decahydronapthalene, triethylbenzene, ethyltoluene, 1-phenylhexane, 1-phenyl-heptane, t-butyl cyclohexane, cyclooctane, and C₁₀-C₁₂ straight or branched saturated hydrocarbons.

12. A method of preparing 2-[(1S)-1-(2R}-oxiranyl-2-propenyl]-1H-isoindole-1,3(2H)-dione comprising recovering (2S,3R)-1,2-epoxy-4-penten-3-ol from an asymmetric epoxidation reaction mixture according to the process of claim 8 and contacting the (2S,3R)-1,2-epoxy-4-penten-3-ol so recovered with phthalimide.

13. A method of making an optically active epoxy alcohol comprising:
contacting an allylic alcohol with an organic hydroperoxide and a transition metal-chiral ligand complex epoxidation catalyst in a reaction solvent under conditions to produce the optically active epoxy alcohol;
adding a reducing agent such that substantially all of the organic hydroperoxide is reduced to produce a reduced epoxidation reaction mixture;
concentrating the reduced epoxidation reaction mixture by removal of at least some of the reaction solvent to produce a concentrated epoxidation reaction product;
separating the concentrated epoxidation reaction product in a mm evaporation unit to form a residue fraction and an optically active epoxy alcohol distillate traction; and
distilling the optically active epoxy alcohol distillate fraction to produce a purified optically active epoxy alcohol.

14. The method of claim 13 further comprising adding an azeotropic solvent prior to or after the adding of the reducing agent, wherein the azeotropic solvent forms an azeotrope with the epoxy alcohol.

15. The method of claim 13 further comprising adding an azeotropic solvent following the concentrating of the reduced epoxidation reaction mixture, wherein the azeoptropic solvent forms an azeotrope with the epoxy alcohol.

16. The method of claim 13 wherein the organic hydroperoxide is cumene hydroperoxide.

17. The method of claim 13 wherein the reducing agent is trimethyl phosphite.

18. The method of claim 13 wherein the optically active epoxy alcohol is (2S,3R)-1,2-epoxy-4-penten-3-ol.

19. The process of claim 18 wherein the reducing agent is trimethyl phosphite and the organic hydroperoxide is cumene hydroperoxide.

20. The process of claim 19 further comprising adding an azeotropic solvent to the concentrated epoxidation reaction product prior to or concurrent with separating the concentrated epoxidation reaction product in the film evaporation unit, wherein the azeotropic solvent forms an azeotrope with the epoxy alcohol.

21. The process of claim 20 wherein the azeotropic solvent is selected from the group consisting of decahydronapthalene, triethylbenzene, ethyltoluene, 1-phenylhexane, 1-phenyl-heptane, t-butyl cyclohexane, cyclooctane, and C₁₀-C₁₂ straight or branched saturated hydrocarbons.

22. A method of preparing 2-[(1S)-1-(2R)-oxiranyl-2-propenyl]-1H-isoindole-1,3(2H)-dione comprising recovering (2S,3R)-1,2-epoxy-4-penten-3-ol from an asymmetric epoxidation reaction mixture according to the process of claim 18 and contacting the (2S,3R)-1,2-epoxy-4-penten-3-ol so recovered with phthalimide.

23. A composition comprising at least 70% by weight of an optically active epoxy alcohol, less than 15% by weight of an azeotropic solvent that forms an azeotrope with the epoxy alcohol, and 0.05% to 2% by weight of cumene alcohol.

24. The composition of claim 23 further comprising trimethyl phosphate.

25. The optically active epoxy alcohol recovered from an asymmetric epoxidation reaction mixture according to the process of claim 1.

26. The optically active epoxy alcohol made by the method of claim 13.
